(19) United States
(12) Patent Application Publication
(10) Pub. No.: US 2004/0243315 A1
(43) Pub. Date: Dec. 2, 2004

(54) Device and process for the assignment of NMR signals of polypeptides

(76) Inventors: Dirk Labudde, Berlin (DE); Dietmar Leitner, Berlin (DE); Mario Schubert, Berlin (DE); Rudiger Winter, Berlin (DE); Hartmut Oschkinat, Berlin (DE); Peter Schmieder, Hohen Neuendorf (DE)

Correspondence Address: Schwegman, Lundberg, Woessner & Kluth, P.A., P.O. Box 2938, Minneapolis, MN, 55402, US

(21) Appl. No.: 10/799,447

(22) Filed: Mar. 11, 2004

Related U.S. Application Data
(63) Continuation of application No. PCT/EP02/09959, filed on Sep. 5, 2002.

(30) Foreign Application Priority Data
Sep. 11, 2001 (DE) 101 44 661.6

(51) Int. Cl. G06F 19/00; G01N 33/48; G01N 33/50

(52) U.S. Cl. 702/19

(57) ABSTRACT

NMR spectroscopy is increasingly used for the determination of the structure of proteins. According to the invention cross-signal pattern search masks are used for the joint evaluation of the 3D triple resonance experiments as well as the amino acid type-specific 2D spectra. All signal peaks due to an amino acid or a group of amino acids whose exact sequence has previously been derived from the protein sequence and forms the basis for the specific pattern recognition can be detected and evaluated jointly by means of pattern recognition with the aid of the cross-signal pattern search masks according to the invention. The invention permits a fully automated assignment of the signal peaks present in the various spectra to the magnetically active nuclei of the protein.

### CROSS-REFERENCE TO RELATED APPLICATIONS

This application is a continuation under 35 U.S.C. 111(a) of PCT/EP02/09959, filed Sep. 5, 2002 and published in English on Mar. 20, 2003 as WO 03/023384 A1, which claims priority from German application No. 101 44 661.6, filed on Sep. 11, 2001, which applications and publication are incorporated herein by reference.

### FIELD OF THE INVENTION

The invention relates to an analysis system for the automated analysis of a set of NMR spectra that has been recorded for a polypeptide chain comprising n amino acids, as well as a process for the automated analysis of a set of NMR spectra.

### BACKROUND OF THE INVENTION

NMR spectroscopy has in recent years established itself as a method for the structure elucidation of small proteins and DNA fragments. NMR spectroscopy allows the investigation of biological macromolecules in solution—with particular regard to dynamic phenomena—and thus constitutes a complementary method to X-ray crystallography.

NMR spectroscopic investigation of proteins was—with few exceptions—initially restricted to relatively small types of proteins with a size of up to 80 amino acids, since for larger proteins its scope is limited by signal overlaps in the two-dimensional spectra. Only the introduction of three-dimensional and four-dimensional NMR techniques (3D- and 4D-NMR) enabled this barrier to be overcome. In conjunction with marking the proteins with ²H, ¹³C and ¹⁵N, nowadays systems with a molecular weight of up to 50 kD can be investigated. The size of the proteins that can still be investigated is basically determined by the transverse relaxation time, which becomes shorter with increasing molecular weight.

The large number of existing multidimensional NMR techniques are necessary within the scope of structure determination projects for two different partial steps. In a first step all ¹H, ¹³C and ¹⁵N signals of a protein have to be assigned. In this assignment step the corresponding signal in the spectrum must be found for these magnetically active nuclei in the protein. Special pulse sequences are available for this assignment task. An overview of the various experiments and pulse sequences employed for the protein structure determination are given in the article “Protein Structure Determination with Three- and Four-Dimensional NMR Spectroscopy” by H. Oschkinat et al., Angew. Chem. Int. Ed. Engl. 1994, 33, pp. 277-293.

In the article “MUSIC, Selective Pulses, and Tuned Delays: Amino Acid Type-Selective ¹H-¹⁵N Correlations, II” by M. Schubert et al., Journal of Magnetic Resonance 148, 2001, pp. 61-72, a number of amino acid type-specific ¹H/¹⁵N experiments are described, in which by utilising the side chain topology the signals of a specific type of amino acid (e.g. Ser) or a specific group of amino acids (e.g. Ile/Val) are contained. The pulse sequences required to carry out these amino acid type-specific 2D experiments can be derived in a simple way from the triple resonance experiments used to determine the structure of the main chain.

After the assignment has been completed, structure parameters of the protein can be collected by means of other NMR techniques. This second step builds on the assignment obtained in the first step. For example, interspacings between various magnetically active nuclei can be determined by means of the various multidimensional versions of the NOESY experiment. The structure parameters thereby obtained serve as input quantities for structure determination software packages. Such structure determination programs generate a three-dimensional model of the polypeptide from the input structure information.

At the present time the various steps of the protein structure analysis are carried out in the various NMR research groups using semi-automated procedures and in most cases in-house software. The many attempts to facilitate in particular the assignment process have led inter alia to so-called electronic plotting tables, in which the spectra are shown on a screen and are assigned with aids provided by the program, such as automatic peak-picking and the possibility of spectral overlap.

Many processes for the automatic assignment of NMR signals are based on the use of cross-signal lists, with which the frequency co-ordinates of the cross-signals are collected. These cross-signal lists can be evaluated with the aid of combinatorial procedures that provide comparisons between the frequencies contained in the cross-signal lists. If the assignment is carried out with the aid of cross-signal lists, a number of disadvantages must be taken into account. Thus, with spectra having a low signal-to-noise ratio or a strong T1 noise, spectral artefacts occur that produce undesirable additional entries in the cross-signal lists and complicate the successful use of the combinatorial procedures. If the cross-signals of the spectrum lie very close together, the individual cross-signals can no longer be resolved with respect to one another. In this case a cross-signal list is obtained in which various entries are missing or are incorrect.

For these reasons the use of cross-signal lists has been abandoned and instead attempts have been made to detect the signal patterns contained in the NMR spectra with the aid of alternative methods. Such an approach is described in the article “Tools for the automated assignment of high-resolution three-dimensional protein NMR spectra based on pattern recognition techniques” by D. Croft et al., Journal of Biomolecular NMR, 10, 1997, pp. 207-219. This article discusses in particular the signal pattern recognition software CATCH23. This software uses search masks for the analysis of the NMR spectra and carries out a pattern search with the help of a combination of search masks. Such a cross-signal pattern search mask covers a plurality of search regions for the anticipated cross-signals of a specific main chain fraction or side chain fraction. For example, all cross-signals due to the amino acid threonine can be detected using a cross-signal pattern search mask. If the cross-signal pattern search mask identifies the corresponding peaks, an assignment can be made between these peaks and the amino acid threonine.

Often however there are several possible ways in which an assignment can be made between the cross-signals on the one hand and the molecular structure on the other hand. This ambiguity in the assignment often necessitates a manual intervention. The cross-signal pattern search masks defined in the published version of the software CATCH23 also does not provide sufficient stability and security for a fully automated assignment of the signal peaks.

Thus, there is a need in the art for a device as well as a process for the automated assignment of the NMR signals of a set of NMR spectra that permits a reliable and unambiguous assignment of the NMR signals to the various magnetically active nuclei and that reduces the number of the necessary manual interventions.

### SUMMARY OF THE INVENTION

The present invention provides a device as well as a process for the automated assignment of the NMR signals of a set of NMR spectra that permits a reliable and unambiguous assignment of the NMR signals to the various magnetically active nuclei and that reduces the number of the necessary manual interventions.

In one embodiment of the invention, a system is provided for the automated analysis of a set of nuclear magnetic resonance (NMR) spectral recordings of a polypeptide comprising a library of cross-signal pattern search masks comprising masks for the specific detection of signals recorded from a fragment of the polypeptide, a selection module adapted to selecting a mask corresponding to the primary sequence of each fragment of the polypeptide, a pattern recognition module adapted to combine the various results of the cross-signal pattern search masks selected and correlate the masks to the set of NMR spectral recordings, and an assignment module adapted to assign the signals to various spin systems corresponding to the primary sequence of the polypeptide.

In another embodiment, the invention provides a process for the automated analysis of a set of NMR spectra, recorded for a polypeptide chain, comprising (a) selecting a cross-signal pattern search mask from a library of cross-signal pattern search masks, wherein the mask detects a NMR signal of a fragment of the polypeptide chain, and wherein the selection of the required cross-signal pattern search masks is made corresponding to the fragments contained in the primary sequence, (b) executing a pattern recognition by correlating the various selected cross-signal pattern search masks with the set of NMR spectra, and (c) assigning the NMR signal to the various spin systems of the polypeptide chain corresponding to the result of the pattern recognition carried out in step b).

### BRIEF DESCRIPTION OF THE DRAWINGS

FIG. 1 is a flow chart for recording the necessary NMR spectra.

FIG. 2 is a table from which the pairs of amino acids contained in the amino acid sequence can be read.

FIG. 3 is a series of examples of amino acid type-specific 2D experiments, by means of which the presence or absence of specific side chain structures can specifically be interrogated.

FIG. 4 is a cross-signal pattern search mask with which the signal patterns contained in the spectra can be recognised and evaluated.

FIGS. 5A and 5B are flow charts for the evaluation of the recorded NMR spectra, in which an assignment is made between the occurring signal peaks and the spin systems of the protein.

### DETAILED DESCRIPTION OF THE INVENTION

The analysis system according to the invention serves for the automated analysis of a set of NMR spectra that has been recorded for a polypeptide chain comprising n amino acids, and comprises a library of cross-signal pattern search masks, in which a cross-signal pattern search mask is provided for the specific detection of the NMR signals of a fragment of the investigated polypeptide chain. In this connection cross-signals of a fragment of an amino acid, or of fragments of several sequentially following amino acids, or all cross-signals of one or more amino acids that are coupled sequentially in the polypeptide chain, can be detected. The fragments may thus consist of bound main chain atoms, possibly including the β carbon atoms, or only of side chain atoms of the individual or coupled amino acids. Furthermore the analysis system comprises means for selecting (e.g., a selection module) the cross-signal pattern search masks of the library corresponding to the primary sequence of the polypeptide chain and required for the analysis, that select the associated cross-signal pattern search masks for each fragment contained in the primary sequence. Also the analysis system has means for pattern recognition (e.g., a pattern recognition module) that combine the various results of the cross-signal pattern search masks selected corresponding to the primary sequence of the polypeptide chain and correlate the results to the set of NMR spectra. Over and above this, the analysis system comprises means for assigning (e.g., an assignment module) the NMR signals to the various spin systems of the polypeptide chain corresponding to the result of the pattern recognition.

The solution according to the invention then permits in particular a very reliable assignment if for a protein a set of NMR spectra is recorded that contains, apart from 3D experiments for the assignment of the main chain signals and side chain signals, also amino acid type-specific 2D experiments. These amino acid type-specific NMR experiments contain only cross-signals of one or more types of amino acids, which either form correlations between signals of atoms in the protein main chain or correlations of side chain signals.

The cross-signal pattern search masks according to the invention are designed so that signal patterns of fragments that belong to specific amino acid types or couplings thereof can be recognised by a combined application to amino acid type-specific NMR experiments and 3D triple resonance experiments. This recognition of the signals of specific amino acid types contained in the fragments is then particularly successful if this is used as starting point at the beginning of the search, in the form of two-dimensional correlations of signals of the protein main chain or of signals of the side chains.

Using the cross-signal pattern search masks according to the invention, in this way in particular the patterns can be interrogated and the fragments that are derived from specific combinations of two and three amino acids, such as for example the pair valine-threonine, can be sought. Such combinations occur only once or a few times in the polypeptide chain, resulting in a correspondingly short target list. This high selectivity of a cross-signal pattern search mask specific for groups of two or three amino acids permits, in the large majority of cases, an unambiguous assignment between the detected cross-signals and the associated fragment of the polypeptide chain. Ambiguities in the assignment can thereby be reduced.

After assignments between the cross-signals on the one hand and the various found fragments on the other hand, have been made with the aid of the cross-signal pattern search masks, these various partial assignments have to be combined in a second step. An assignment of all the signals of the spectrum to the associated magnetically active nuclei of the polypeptide chain is achieved by combining the various partial assignments. By using the cross-signal pattern search masks according to the invention to detect fragments of the investigated polypeptide chain, there is obtained in each case an overlap of the various fragments to be evaluated. For the magnetically active nuclei in the overlap region the chemical shifts determined in each case with different cross-signal pattern search masks must coincide. The various partial assignments can be combined with the aid of this boundary condition, attention being concentrated in particular on the chemical shifts of H_{N} as well as N. On account of the overlap between the cross-signal pattern search masks the combination of the partial assignments obtained from selective searches to form an overall assignment can be accomplished in a substantially simpler and more reliable manner with the cross-signal pattern search masks according to the invention than was possible in the prior art. The cross-signal pattern search masks according to the invention offer advantages, both in the actual peak assignment as well as in the subsequent combination of the partial assignments, compared to the simple cross-signal pattern search masks of the prior art.

Overall a higher reliability in the assignment as well as a better recognition rate is made possible by the use of the cross-signal pattern search masks according to the invention, with which the NMR signals of a fragment of the investigated polypeptide chain can be specifically detected. Since the number of ambiguities arising in the assignment is reduced, fewer manual interventions have to be made in the course of the assignment. To this extent the invention represents an important step in the transition from a semi-automated to a fully automated assignment of NMR signal peaks. Once a reliable, fully automated assignment is possible, the throughput in the determination of protein structures can be significantly raised. Also, the reliability of the structural data that are thus obtained is improved.

In addition an ingenious and instructive encapsulation of the analysis tools for the assignment of the cross-signal patterns is achieved with the aid of the cross-signal pattern search masks according to the invention. The results of the assignment can thereby also more easily be reproduced.

It is an advantage if the fragments of the investigated polypeptide chain in each case comprise two or three specific, sequentially contiguous amino acids. A specific fragment consisting of two (or three) amino acids can be identified unambiguously on the basis of its NMR signals using a cross-signal pattern search mask according to the invention. Using the cross-signal pattern search masks according to the invention, in this way in particular the patterns can be interrogated and the fragments that are derived from specific combinations of two and three amino acids, such as for example the pair valine-threonine, can be sought. Such combinations occur only once or a few times in the polypeptide chain, resulting in a correspondingly short target list. This high selectivity of a cross-signal pattern search mask specific for groups of two or three amino acids permits in the overwhelming majority of cases an unambiguous assignment between the detected cross-signals and the associated fragment of the polypeptide chain. Ambiguities in the assignment can thereby be reduced. Thus, in one embodiment of the invention, “n” is two or three.

It is advantageous if the set of NMR spectra includes NMR experiments on the analysis of the main chain signals as well as NMR experiments on the analysis of the side chain signals. The coupling between the side chains and the main chain is achieved in particular via the chemical shift of the C_{α} nuclei as well as of the C_{β} nuclei. The main chain signals and side chain signals can be evaluated jointly with the aid of the cross-signal pattern search masks according to the invention, which are provided for the specific detection of the NMR signals of a fragment of the investigated polypeptide chain.

It is advantageous if the NMR experiments used for the analysis of the main chain signals include 3D experiments and in particular 3D experiments of the types CBCA(CO)NNH, CBCANNH, HA(CO)NNH, HANNH, HAHB(CO)NNH, HAHBNNH, HN(CA)CO, HNCO, HN(CO)CA, HNCA. The listed experiments involve 3D experiments by means of which the chemical shifts of the magnetically active nuclei of the main chain can be detected. The large number of available 3D experiments also allows however a multiple confirmation of the results.

It is advantageous if the NMR experiments used for the analysis of the side chain signals include experiments of the types HCCH-COSY, HCCH-TOCSY, HCC(CO)NH-TOCSY.

It is advantageous if the NMR experiments used for the analysis of the main chain signals and side chain signals include amino acid type-specific ¹H/¹⁵N experiments that are selective for an amino acid type or for a group of amino acid types. For a protein a set of NMR spectra is recorded that contains, apart from the 3D experiments for detecting the main chain structure, also amino acid type-specific 2D experiments. These amino acid type-specific NMR experiments contain only cross-signals of one or more amino acid types, which represent either correlations between signals of atoms in the protein main chain or correlations of side chain signals. Amino acid type-specific 2D experiments permit the selective excitation of the side chains of an amino acid type or of a group of amino acid types. The magnetisation is then transferred via the side chain to the main chain nitrogen atoms and amide protons. The NMR signals caused by a specific amino acid type or a group of amino acid types, which constitute a type of “fingerprint” of a specific amino acid type or group of amino acid types, can be detected in a highly specific manner with the aid of amino acid type-specific ¹H/¹⁵N experiments. The NMR signals of a fragment of the investigated polypeptide chain can then be interrogated in a targeted manner with a cross-signal pattern search mask according to the invention. A higher reliability in the assignment as well as better recognition rate is made possible in this way.

The pulse sequences required for carrying out the amino acid type-specific 2D experiments can be derived in a simple way from the triple resonance experiments used in particular to determine the main chain structure.

It is advantageous if the amino acid type-specific 2D experiments required for the analysis of the main chain signals and side chain signals are specified corresponding to the primary sequence of the polypeptide chain. This primary sequence is known beforehand. The amount of protein required to carry out the NMR measurements is in fact produced by means of biotechnology methods with the assistance of a corresponding DNA sequence. If now for example the amino acid cysteine does not occur in the primary sequence of the polypeptide chain, it is also not necessary to carry out the amino acid type-specific 2D experiment for cysteine. The minimum necessary set of data for the NMR experiments can thus be specified on the basis of the primary sequence.

It is advantageous if the NMR experiments used for the analysis of the main chain signals and side chain signals include a combination of 2D and 3D experiments. The combined use of main chain experiments and amino acid type-specific 2D experiments, together with the evaluation of the NMR signals with the help of cross-signal pattern search masks, permits a considerable performance enhancement in the automated evaluation of NMR spectra. The cross-signal pattern search masks according to the invention are designed so that signal patterns of fragments that belong to specific amino acid types or couplings thereof can be recognised by a combined application to amino acid type-specific NMR experiments and 3D triple resonance experiments. Since the number of ambiguities arising in the assignment can be reduced in particular with the aid of amino acid type-specific 2D experiments, fewer manual interventions have to be made in the course of the assignment. The invention thus constitutes an important step in the transition from a semi-automated to a fully automated evaluation of NMR spectra.

According to an advantageous embodiment of the invention, starting from the assignment of the NMR signals to the various spin systems of the polypeptide chain the chemical shifts are combined and checked for their correctness. Starting from the assignment of the NMR signals, the chemical shifts determined for the various magnetically active nuclei of an amino acid may for example be combined in the form of a vector. A consistency check may then be carried out with respect to the main chain, in particular by means of the chemical shifts of H_{N} and N that are determined independently of one another in different experiments. Coincident or closely adjacent values must be obtained for the chemical shifts determined in different experiments. The chemical shifts of the C_{α} and C_{β} nuclei that are determined in main chain experiments as well as in side chain experiments accordingly permit such a consistency check to be performed for the main and side chains.

According to a further embodiment of the invention the set of NMR spectra comprises spectra of the NOESY type, whose evaluation provides in particular information on the distances of the various nuclei of the polypeptide chain. In experiments of the NOESY type the cross-relaxation due to the Kern-Overhauser effect is detected. Feedback on the distances between the nuclei involved can be obtained from the amplitudes of the NOE cross-signals. NOESY type spectra are therefore particularly important for determining protein structure.

According to a further embodiment of the invention the NMR spectra of the NOESY type are assigned to the various nuclei of the polypeptide chain on the basis of the chemical shifts determined for the nuclei. The assignment of the cross-signals in the NOESY spectra to the various magnetically active nuclei is carried out in particular on the basis of the proton chemical shifts. However, even if the proton chemical shifts have been determined beforehand with sufficient accuracy and are therefore already known, ambiguities still remain due to the multiple denotation of the individual cross-signals. For this reason it is all the more important to be able to assign unambiguously as large a proportion of the NOESY spectra as possible on the basis of proton chemical shifts that have been determined as accurately as possible.

According to a further advantageous embodiment of the invention the values obtained in the evaluation of the NMR spectra serve as input quantities for structure calculation software. Important input quantities for structure calculation programs are in particular the distances of the nuclei obtained from the assigned NOESY spectra. For this purpose a list of the amplitudes of the NOE cross-signals and of the frequency co-ordinates of the peaks as well as the resonance assignment can be used for the structure calculation program. Further input quantities may include the coupling constants between different nuclei, since from these coupling constants the dihedral angles between different nuclei can be determined.

It is advantageous if the cross-signal pattern search masks in each case comprise a number of predefined signal search regions, in which due to the occurrence of NMR signals within the region boundaries of a signal search region there is an increased probability that the signal pattern defined by the cross-signal pattern search mask is present. In this way the peak pattern to be sought can be defined exactly by means of a number of search regions. A true pattern recognition thereby becomes possible. Each signal peak occurring within the boundaries of a search region increases the evaluation score for the cross-signal pattern search mask. In this connection it is particularly advantageous that even if individual peaks of the signal pattern are missing, a signal pattern can still be recognised if it otherwise agrees sufficiently well with the cross-signal pattern search mask. When evaluating whether a peak pattern agrees with the cross-signal pattern search mask, the important factor is the overall established agreements.

In this connection it is advantageous if the cross-signal pattern search masks in each case comprise a number of predefined empty regions, whereby due to the absence of NMR signals within the region boundaries of an empty region there is an increased probability that the signal pattern defined by the cross-signal pattern search mask is present. The definition of empty regions is then meaningful for example if two different side chain structures lead to two signal patterns that are similar to one another, the second signal pattern having some additional peaks that are not contained in the first signal pattern. The absence of these signal peaks is then exactly that typical of the first signal pattern, which means that in order to detect the first signal pattern it is recommended to define empty regions at the corresponding sites. If then no peaks occur within the boundaries of the empty regions, the evaluation score for the presence of the first signal pattern is thus increased. The two signal patterns can accordingly be better differentiated by the definition of empty regions.

It is advantageous if, starting from the expected number of NMR signals in the spectra, the threshold values and search regions for the cross-signal pattern search masks are determined by iteration. In this procedure the search regions are defined at the start by widely set boundaries, whereas the threshold values are chosen relatively low. The evaluation of the signal peaks found in the first run provides initial predictions of which cross-signal pattern or patterns are likely to be present. In a second, modified search an attempt can then be made specifically to find these most probable candidates, in which connection the search regions are reduced or displaced, depending on the chemical shifts of the peaks found in the first search, in order to refine the search. It is also possible to operate with increased threshold values in the second search. By means of this iterative procedure the cross-signal pattern search can be caused to converge stepwise in the direction of the actually present cross-signal patterns.

According to a further advantageous embodiment of the invention the cross-signal pattern search mask comprises a plurality of sub-search masks for analysing the various NMR spectra of the recorded set of NMR spectra. The actual cross-signal pattern search mask arises in this connection as the totality of different sub-search masks that in each case search different two-dimensional, three-dimensional or higher dimensional spectra. The set of NMR spectra is thus analysed with a corresponding set of sub-search masks. This has in particular the advantage that the modification of search region boundaries acts simultaneously on all sub-search masks. The handling of the set of search masks is thereby simplified.

In the process according to the invention for the automated analysis of a set of NMR spectra that has been recorded for a polypeptide chain comprising n amino acids, the cross-signal pattern search masks required for the analysis are first of all selected from a library of cross-signal pattern search masks, in which a cross-signal pattern search mask is provided for the specific detection of the NMR signals of a fragment of the investigated polypeptide chain, and in which the selection of the necessary cross-signal pattern search masks is made corresponding to the fragments contained in the primary sequence. A pattern recognition is then carried out by correlating the different selected cross-signal pattern search masks with the set of NMR spectra. The NMR signals are assigned to the different spin systems of the polypeptide chain corresponding to the result of this pattern recognition.

Since the cross-signal pattern search masks according to the invention in each case jointly evaluate all NMR signals of a fragment of the investigated polypeptide chain, the NMR signals of a fragment, for example, of a fragment comprising two, three or more amino acids, can be detected in a highly selective manner by means of the process according to the invention. If then reliable signal assignments exist for the individual fragments of the polypeptide chain, an overall assignment of the NMR signals of the polypeptide chain can be derived on account of the overlap between the analysis results. The unambiguity and reliability of the assignments is improved compared to the processes of the prior art. Manual interventions by the user are required less often and for this reason the process is suitable in particular for the fully automated evaluation of NMR spectra. Time and cost need involved in the determination of protein structures by NMR spectroscopy can thereby be reduced further.

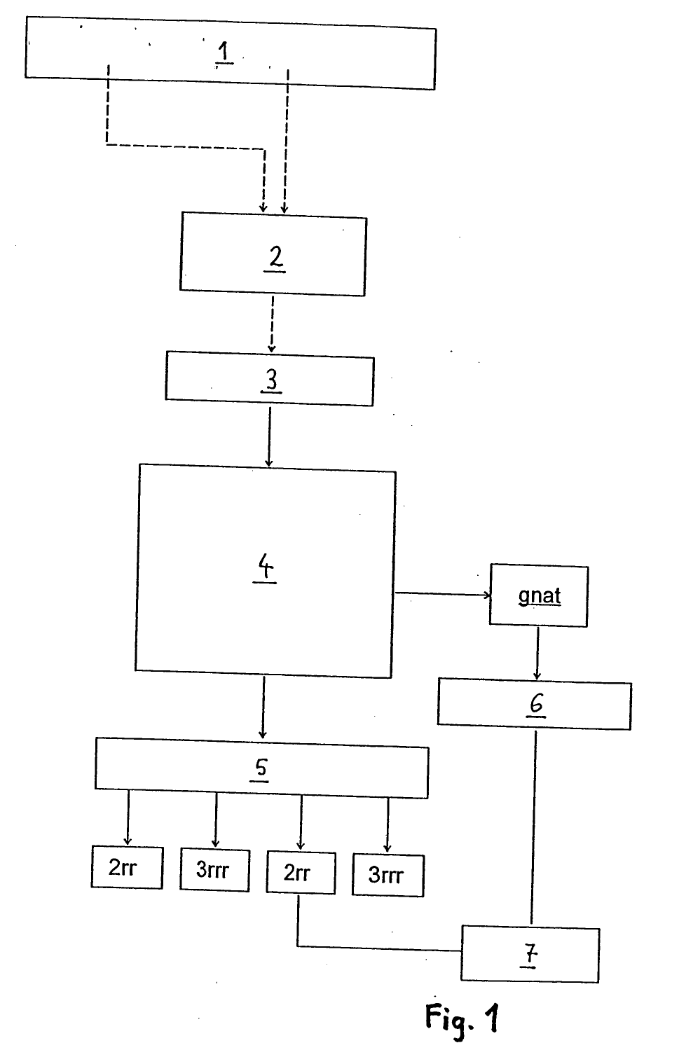

The invention is described in more detail hereinafter with the aid of an example of implementation illustrated in the drawings, in which FIG. 1 illustrates the flow chart used to record the necessary NMR spectra. The starting point for deciding the necessary experiments is the primary structure of the protein. In most cases the required proteins are synthesised by means of biotechnology methods with the aid of corresponding DNA sections, since the required amounts of proteins can easily be produced in this way. It is therefore assumed in the following description that the primary structure of the protein is known beforehand, and that the NMR spectroscopy experiments should be used simply to determine the structure of the protein.

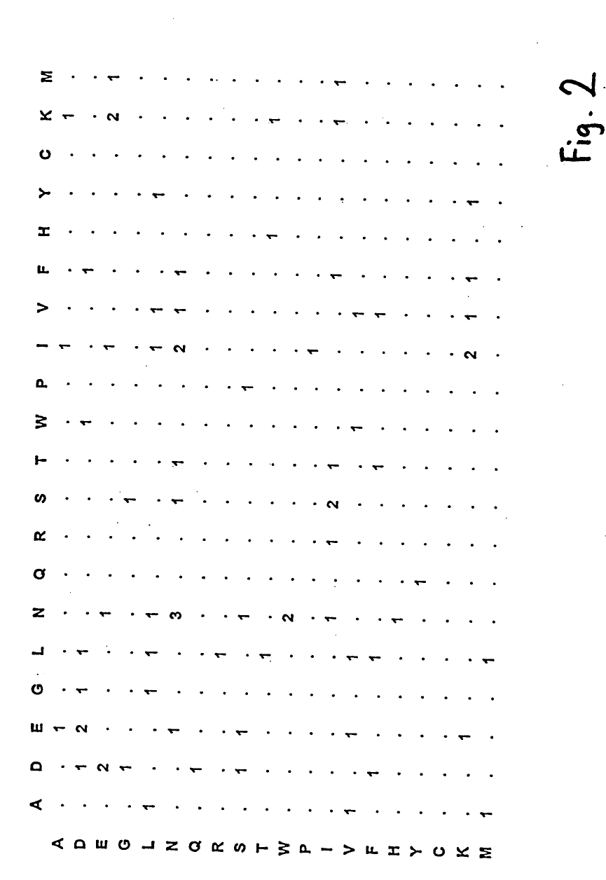

In step 1 the pairs of successive amino acids contained in the primary sequence are determined and listed. Since the primary structure is known, this can be carried out in a very simple way by a self-written computer program by the name of “selma”. The result supplied by the “selma” program for the protein OPR is shown in FIG. 2. The letters plotted along the x axis and the y axis denote in each case the 20 possible amino acids. The amino acids listed along the y axis denote the amino acid present in the first position of the amino acid pair in question, while the amino acids listed along the x axis denote the amino acid at the second position of the amino acid pair.

The various amino acid pairs occurring in the amino acid sequence of the protein OPR are entered in the resulting matrix. It can be seen from the table that the amino acid pair AE occurs precisely once in the sequence, whereas the amino acid pair EA is not contained in the sequence. Where the number 2 or 3 is entered at a specific matrix position, this means that the corresponding amino acid pair occurs more than once in the sequence. This is the case for example with the amino acid pair ED.

The information thereby obtained on the amino acid pairs contained in the primary sequence serves in step 2 to specify a set of NMR experiments that have to be carried out in order to determine the protein structure. The aim is to perform as few superfluous experiments as possible, which would only unnecessarily prolong the measurement time. Thus, it can be seen for example on the basis of the results of the “selma” program shown in FIG. 2 that the amino acid cysteine is not present in the protein OPR. It is not necessary to record a cysteine-selective 2D experiment, and the cysteine-selective 2D experiment is therefore also not part of the set of NMR experiments specified in step 2.

In step 3 the two-dimensional or multidimensional NMR spectra of the set are recorded in an NMR spectrometer. The spectrometer comprises a spectrometer control device, which initially determines and adjusts the operating parameters of the spectrometer, such as for example the proton carrier frequency as well as the length of 90° pulses. The spectra specified in step 2 are then recorded in succession. To this end the spectrometer control device contains a selection of standardised NMR pulse sequences.

The recording of the NMR spectra necessary for a protein requires a measurement time of several weeks. The recorded spectra are then available as datasets of a project sequencer 4 and may be evaluated further in step 5. 2D spectra “2rr” as well as 3D spectra “3rrr” are obtained as a result. From the datasets contained in the project sequencer 4, the “gnat” program determines various statistical parameters 6 needed for the further evaluation, which are required for example to determine threshold values. With the aid of such threshold values the peaks contained in the spectra can be differentiated from background noise.

In order to record the chemical shifts of the magnetically active nuclei of the main chain, typically 3D triple resonance experiments as well as amino acid type-specific 2D experiments are used, with which in particular also the resonances of ¹³C and ¹⁵N nuclei can be detected. In particular the following pairs of 3D experiments may be used for this purpose: CBCA(CO)NNH and CBCANNH, HA(CO)NNH and HANNH, HAHB(CO)NNH and HAHBNNH, HN(CA)CO and HNCO as well as HN(CO)CA and HNCA. Nuclei given in brackets (e.g. 3D-HN(CO)CA) are not detected, but are involved in a coherence transfer.

In order to determine the necessary chemical shifts it would already be sufficient to carry out a small number of triple resonance experiments. In order to obtain reliable results it is however advantageous to record the different correlations occurring in the protein in each case by means of several experiments so that the results can be checked for their consistency.

By means of the CACBNNH experiment the frequencies of the H_{N}, N, C_{α} and C_{β} nuclei of the ith amino acid as well as the frequencies of the C_{α} and C_{β} nuclei of the amino acid i−1 are obtained. The CACB(CO)NH spectrum contains the correlations of the H_{N} and N nuclei of the amino acid i with the C_{α} and C_{β} nuclei of the amino acid i−1.

The frequencies of the N, H_{N} and C_{α} nuclei of the ith amino acid are detected with the 3D experiment of the HNCA type. The frequencies of the N, H_{N} as well as C_{α} nuclei of the amino acid i+1 can also be detected in a corresponding way by means of the HNCA experiment. It is furthermore also possible to correlate the C_{α} nucleus off the amino acid i with the N and H_{N} nuclei of the amino acid i+1 with the HNCA experiment.

The basic advantage of the triple resonance experiments is the relatively small number of signal peaks in the spectra. Triple resonance experiments contain per amino acid only one or at most two cross-signals. A large degree of automation of the evaluation thereby becomes possible.

The frequency-specific assignment thus takes place by the combination of individual “building blocks” that intersect in parts. Particularly important are those experiments that permit a detection of the chemical shifts of the β carbon atoms.

The use of the pulse sequence MUSIC (Multiplicity Selective In-Phase Coherence Transfer) has proved particularly advantageous for the clarification of the resonance assignment. MUSIC pulse sequences for the specific excitation of the side chains of a group of amino acids or of a specific type of amino acid may be obtained by modifying the pulse sequences of 3D triple resonance experiments (such as for example CBCA(CO)NNH).

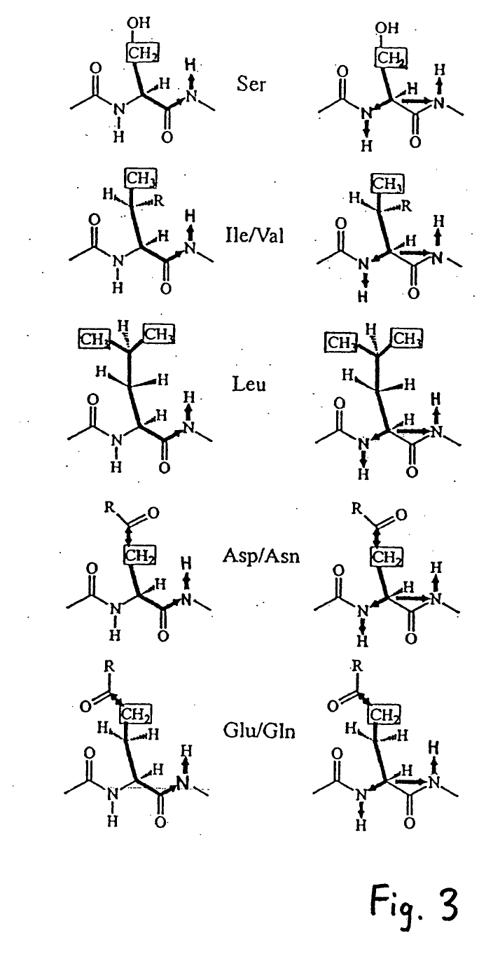

The respective magnetisation transfer for a series of amino acid type-specific 2D experiments is illustrated in FIG. 3. First of all a specific group situated in the side chain is excited by the MUSIC sequence. In the illustrated examples the CH₂ or CH₃ group shown outlined in a rectangle is in each case excited. From there the magnetisation is transferred along the side chain to the C_{α} atom and thence to the N amide proton. The difference between the experiments listed in the left-hand and right-hand column consists in the nature of the transfer from the C_{α} atom to the nitrogen N. In the experiments shown in the left-hand column the magnetisation passes from the C_{α} nucleus to the carbonyl group and from there to the nitrogen N and to the amide proton H_{N} of the amino acid i+1. On account of this magnetisation transfer to the next successive amino acid, these experiments are termed (i+1)-HSQCs. With the (i,i+1)-HSQCs shown in the right-hand column on the other hand the magnetisation passes from the Ca nucleus either to the nitrogen N of the same amino acid i or to the nitrogen N of the adjacent amino acid i+1. By means of the experiments shown in FIG. 3 2D spectra can be selectively recorded for a specific type of amino acid (e.g. for Ser, 1^{st} line; Leu, 3^{rd} line) or selectively for specific groups of amino acids (c.f. Ile/Val, 2^{nd} line; Asp/Asn, 4^{th} line as well as Glu/Gln, 5^{th} line).

The recorded 2D spectra as well as 3D spectra then undergo a pattern recognition in order to be able to assign the signal peaks occurring in the spectra to the individual spin systems of the protein. In the prior art solutions this assignment was generally carried out with the aid of peak signal lists. Compared to such solution approaches, the use of pattern recognition routines offers advantages inasmuch as the cross-signal patterns measured in this case can be evaluated in their totality.

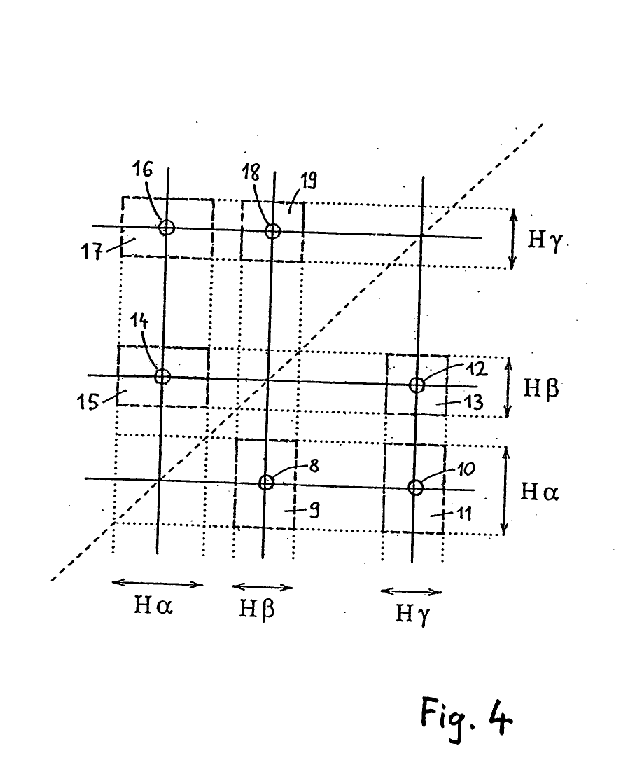

A cross-signal pattern search mask used for the analysis of cross-signal patterns is shown in FIG. 4. Signal peaks are expected at the positions 8, 10, 12 as well as at the mirror image positions 14, 16 and 18 of the two-dimensional spectrum. Rectangular signal search regions 8, 11, 13 as well as 15, 17, 19 are defined around the expected peak positions. Signal peaks occurring within the thus-defined regions are detected by the pattern recognition software, whereas peaks occurring outside the signal search regions are not detected. The cross-signal pattern search mask thus covers the predefined signal search regions 9, 11, 13, 15, 17, 19, the software searching within these signal search regions for the expected signal peaks.

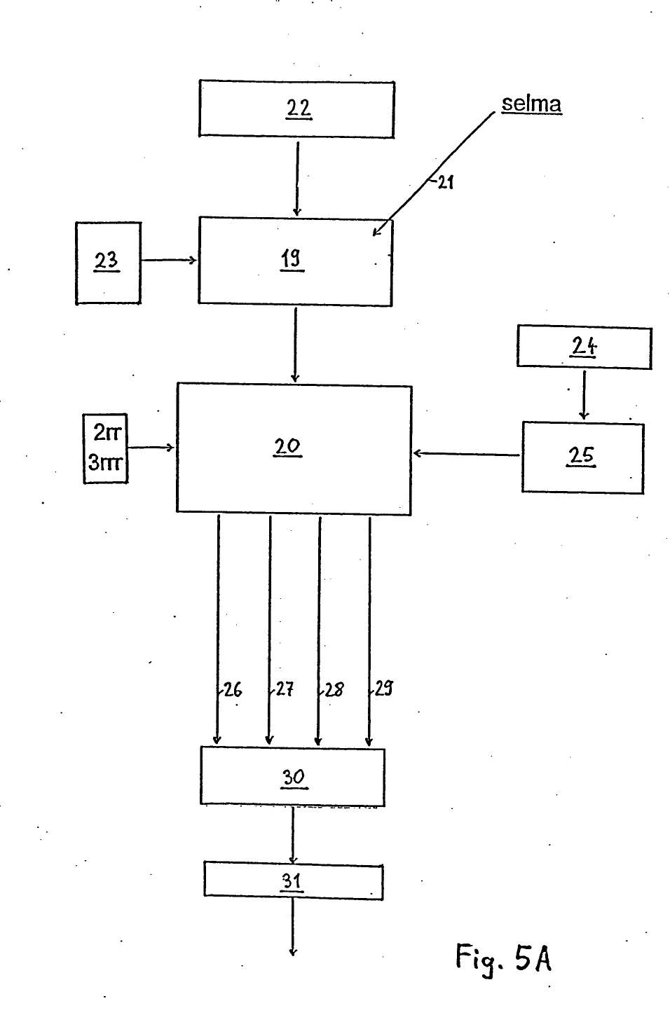

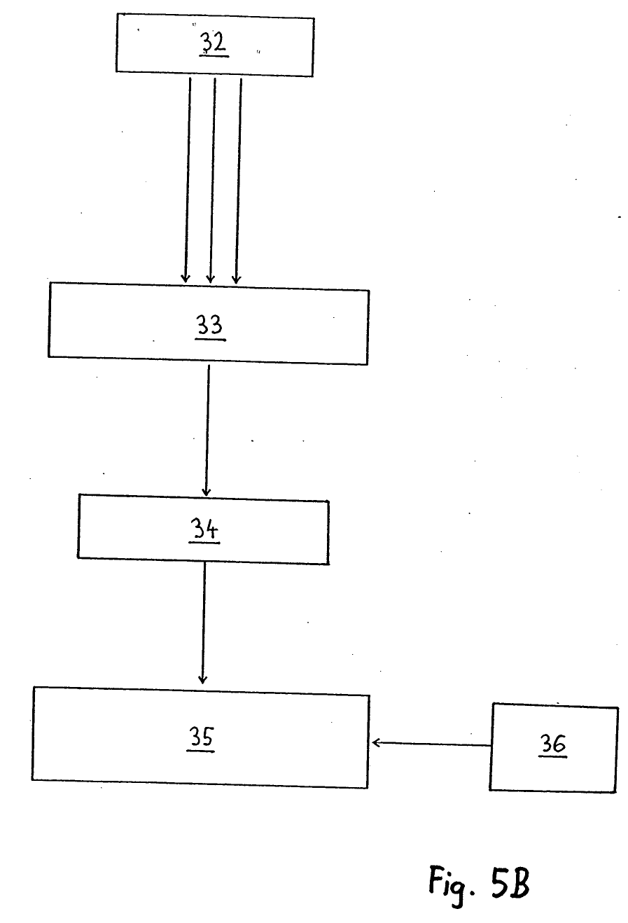

The flow chart of the pattern recognition and assignment is shown in FIGS. 5A and 5B. A library 19 of cross-signal pattern search masks according to the invention that is available to the pattern recognition software 20 serves for the analysis of the cross-signal patterns. The signal peaks of a fragment of two (or three) successive amino acids can be detected and assigned with each of the cross-signal pattern search masks according to the invention. The selection of the cross-signal pattern search masks required for the pattern recognition is made according to the breakdown of the amino acid sequence into two types of fragments that is carried out by the “selma” program. The cross-signal pattern search masks required for the assignment of the signal peaks of the protein are selected from the library 19 corresponding to the two groups contained in the primary sequence and are made available to the pattern recognition software 20.

All signal peaks that are due to a group of two successively arranged amino acids, i.e. the signal peaks due to the two side chains as well as the signal peaks due to the main chain fragment, can be evaluated jointly with the aid of a predefined cross-signal pattern search mask.

It will be assumed in the following description that three different pairs of triple resonance experiments, namely the pairs CBCANNH/CBCA(CO)NNH, HNCO/HN(CA)CO as well as HANNH/HA(CO)NNH, and the amino acid type-specific 2D experiments, are used for the assignment of the main chain signals. In this case the library 19 of cross-signal pattern search masks contains a total of 3×20×20=3×400=1200 different cross-signal pattern search masks, namely

400 cross-signal pattern search masks for the analysis of:

CBCANNH+CBCA(CO)NNH+two amino acid type-specific 2D experiments,

400 cross-signal pattern search masks for the analysis of:

HNCO+HN(CA)CO+two amino acid type-specific 2D experiments, as well as

400 cross-signal pattern search masks for the analysis of:

HANNH+HA(CO)NNH+two amino acid type-specific 2D experiments.

Each of the cross-signal pattern search masks according to the invention serves for the evaluation of two amino acid type-specific 2D experiments as well as a pair of 3D triple resonance experiments. In order to be able to evaluate the different spectra with a cross-signal pattern search mask, the cross-signal pattern search mask comprises a set of sub-search masks, whereby a specific type of spectra can be evaluated with each sub-search mask. From the programming aspect however the cross-signal pattern search mask is presented as a unit. It is therefore possible for the overall cross-signal pattern search mask together with all its sub-search masks to change a specific evaluation parameter, for example a search region boundary. The change then acts in a self-consistent way and manner on the search region boundaries in all sub-search masks.

The predefined cross-signal pattern search masks contained in the library 19 specify the search algorithm for finding specific signal patterns. However, the cross-signal pattern search masks are presented in a parameter-independent form. The necessary search region boundaries 22 as well as the threshold values 23 required to differentiate the signal peaks from the background noise are made available externally to the cross-signal pattern search masks.

This procedure provides the possibility of altering in the course of the search the parameters and search regions that are used to carry out a cross-signal pattern search and of adapting them to newly-obtained information. In particular it is advantageous to set the search region boundaries very wide to start with and to reduce them iteratively depending on the peaks occurring within the search regions, in order thereby to be able to detect the sought cross-signal pattern with a greater degree of certainty. Similarly the threshold values 23 can be raised during the course of the search from an initially low value to increasingly higher values in order thereby to filter out the cross-signal patterns with the highest evaluation scores.

The program routine described hereinafter represents an implementation of a cross-signal pattern search mask that evaluates the two side chain-specific 2D experiments “sHSQCcoN” as well as “sHSQCcaS” and also the pair of 3D triple resonance experiments “HNcoCACB” and “HNCACB”. This cross-signal pattern search mask specific for a pair of amino acids thus comprises four sub-search masks for the evaluation of the various 2D spectra and 3D spectra. The results found by evaluating the various spectra are combined by means of the chemical shifts of the nuclei in the overlap region, i.e. in particular via the chemical shifts of H_{N}, N, C_{α} and C_{β}.

(----------Appendix 1----------)

From the program code it can be seen in particular that the cross-signal pattern search mask in its abstractly defined form also does not have any numerically specified search regions and threshold values. The corresponding variables “submatrix_sizes”, “sweep_widths”, “ppm_offsets” as well as “nucleus_species” are simply defined in abstract form.

The following program listing shows the cross-signal pattern search mask for the 2D spectra “sHSQCcoN”, “sHSQCcaS” as well as for the 3D spectra “HNcoCACB”, “HNCACB”, in which the search region boundaries 22 as well as the threshold values 23 have been prepared in the meantime:

(----------Appendix 2----------)

In particular with regard to the parameters “matrix_sizes”, “submatrix_sizes”, “sweep_widths”, “ppm_offsets”, “nucleus_species” as well as “mask_lower_threshold”, the numerical value range is now defined in each case.

As soon as the search region boundaries 22 as well as the threshold values 23 have been defined, the pattern recognition software 20 can start the actual pattern recognition. For this purpose the recorded 2D spectra “2rr” as well as the 3D spectra “3rrr” are correlated with the search regions of the cross-signal pattern search mask (or one of its sub-search masks), in order to obtain an evaluation score for the presence of the cross-signal pattern detected by the cross-signal pattern search mask.

In the example illustrated in FIG. 4 the values of the spectrum that lie within the signal search regions 9, 11, 13, 15, 17, 19 are summated in order thereby to obtain the evaluation score for the presence of the cross-signal pattern. If the expected signal peaks occur within the signal search regions 9, 11, 13, 15, 17, 19, then a high evaluation score is obtained for the sought cross-signal pattern. This means that the sought cross-signal pattern is present with a high degree of probability. If on the other hand the expected peaks are wholly or partly missing in the signal search regions 9, 11, 13, 15, 17, 19, then a correspondingly low evaluation score is obtained. In this case it is unlikely that the sought cross-signal pattern is present.

In order to calculate the evaluation score a so-called mask scan 24 is performed, in which the co-ordinates of the chemical shift are successively incremented in the different spatial directions in order thereby to raster scan the whole spectrum. The co-ordinate value that is thus generated is compared with the mask data 25. If the co-ordinate value lies outside all the signal search regions of the cross-signal pattern search masks, then the evaluation score remains unchanged. If on the other hand the newly-generated co-ordinate value lies inside a search region, then the spectral value belonging to this co-ordinate value is added to the evaluation score. With the aid of such a mask scan 24 the evaluation score for a specific cross-signal pattern search mask or for a specific sub-search mask of the cross-signal pattern search mask can be determined quickly and simply.

Up to now it has been assumed that the cross-signal pattern search mask simply comprises a number of signal search regions, in which the occurrence of signal peaks is expected within the search region boundaries. However, empty regions may also be correspondingly defined, in which the occurrence of a signal peak within the empty region boundaries leads to a reduction of the evaluation score. Such empty regions constitute, as it were, “forbidden” regions in which no signal peaks may occur.

In order to improve the recognition accuracy an attempt may be made to improve by means of a convolution operation the quality of the 2D and 3D spectra before carrying out the pattern recognition. For this purpose an ideal Gauss signal, whose magnitude and extent roughly corresponds to the magnitude and extent of the expected NMR signal peaks, may be convoluted with the spectrum. In this way artefacts can be suppressed and smudged peaks can be resolved more easily.

FIG. 5A shows how the evaluation of two amino acid type-specific 2D spectra as well as a pair of triple resonance experiments yields four partial results 26, 27, 28, 29. In order to evaluate the two amino acid type-specific 2D spectra two sub-search masks suitable for this purpose may be provided, and to evaluate the two triple resonance experiments two further sub-search masks of the cross-signal pattern search mask may be defined. Each sub-search mask of the cross-signal pattern search mask in question generates a partial result 26, 27, 28, 29. For example, a partial result for a specific 2D spectrum contains the chemical shifts of the peaks found within the signal search regions together with the evaluation score for the sub-search mask.

The four partial results 26, 27, 28, 29 that are obtained in the evaluation of the two amino acid type-specific 2D spectra as well as of the pair of triple resonance experiments are fed to the merging unit 30. The purpose of the merging unit 30 is to combine the different partial results into a result list 31. To this end the chemical shifts in the overlap regions of the individual partial results are compared. The merging, i.e. a combination of the partial results into the result list 31, can then be carried out in particular on the basis of the chemical shifts of the H_{N} as well as N nuclei over an interval of H_{N}±ΔH_{N} as well as of N±ΔN. An entry in the result list 31 comprises a so-called shift vector, in which the chemical shifts occurring within two successive amino acids are listed, as well as the evaluation score determined as a whole for the presence of the group of two amino acids.

Following this the result list that is thus formed is weighted. The weighting procedure is carried out by the cleaning unit 32. The cleaning unit 32 checks the plausibility of the found result by checking the correctness of the shift vectors with a weighting function.

The result list of a cross-signal pattern search mask that searches for the cross-signal patterns due to the amino acid pair N—S is specified hereinafter. As was only to be expected on account of the fragmentation of the primary sequence carried out by means of the “selma” program, simply one entry was found since the group of two successive amino acids N—S in the primary sequence of the protein OPR in question occurs only once. The entry in the result list contains a listing of chemical shifts as well as an evaluation score, which is given under the heading “Resp”.

(----------Appendix 3----------)

In order to check the plausibility of the result list found for the amino acid pair N—S, a pattern search is also carried out in the 3D experiments recorded for the complete protein main chain. In this way a more detailed result list is obtained whose inputs in turn comprise a number of chemical shifts as well as an evaluation score:

(----------Appendix 4----------)

On the basis of the matching chemical shifts it can be seen that the entry #53 is the entry for the amino acid pair N—S. This confirms the consistency of the results found in the two pattern searches.

After partial assignments have been carried out for the individual amino acid pairs contained in the protein sequence, these partial assignments must be copied onto the primary sequence. This step is termed sequence mapping 33. On the basis of the primary sequence the amino acid pairs are searched in the result list and copied, starting with the highest weighting, onto the sequence. After each iteration the chaining of the individual pairs is checked and in this way fragments of 2, 3, 4, etc. amino acids are formed. After completion of the iteration routine the missing fragments are searched in the result lists of the pattern search for the 3D experiment pairs and copied onto the sequence. After completion of the main chain search a targeted search is carried out in the result lists of all side chain experiments.

A complete sequential assignment 34 of the signal peaks occurring in the various spectra to the various amino acids of the sequence is obtained as the result of the sequence mapping 33. The object of achieving as automated an assignment as possible of the spectral peaks is thereby effected.

This assignment as well as the chemical shifts found for the various magnetically active nuclei of the protein may be taken as the starting point for the automated assignment of NOESY spectra 36. This assignment of the NOESY spectra 36 to the various magnetically active nuclei of the protein is executed by the ARIA 35 program. The term “ARIA” stands for “Ambiguous Restraints for Iterative Assignment”. The starting point for ARIA is an almost complete assignment of the proton chemical shifts, which is transmitted together with a list of the amplitudes of the NOE cross-signals and their frequency co-ordinates to a structure calculation program (in the special case “Explor”). The central task of the ARIA 35 program is the assignment of the NOE during the structure calculation by adopting a multiple meaning of the individual cross-signals and using an iterative assignment strategy for the latter.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A system for the automated analysis of a set of nuclear magnetic resonance (NMR) spectral recordings of a polypeptide comprising:
(a) a library of cross-signal pattern search masks comprising masks for the specific detection of signals recorded from a fragment of the polypeptide;
(b) a selection module adapted to selecting a mask corresponding to the primary sequence of each fragment of the polypeptide;
(c) a pattern recognition module adapted to combine the various results of the cross-signal pattern search masks selected and correlate the masks to the set of NMR spectral recordings; and
(d) an assignment module adapted to assign the signals to various spin systems corresponding to the primary sequence of the polypeptide.

2. The system of claim 1, wherein the fragment comprises two or three contiguous amino acids.

3. The system of claim 1, wherein the set of NMR spectra comprises NMR experiments for the analysis of main chain signals and NMR experiments for the analysis of side chain signals.

4. The system of claim 3, wherein the NMR experiments for the analysis of the main chain signals comprise 3D experiments.

5. The system of claim 4, wherein the 3D experiments comprise CBCA (CO)NNH, CBCANNH, HA (CO)NNH, HANNH, HAHB (CO)NNH, HAHBNNH, HN (CA) CO, HNCO, HN(CO) CA and HNCA type experiments.

6. The system of claim 3, wherein the NMR experiments for the analysis of the side chain signals comprise HCCH-COSY, HCCH-TOCSY or HCC(CO)NH-TOCSY type experiments.

7. The system of claim 3, wherein the NMR experiments for the analysis of the main chain signals and side chain signals comprise amino acid type-specific 2D experiments that are selective for an amino acid type or for a group of amino acid types.

8. The system of claim 7, wherein the amino acid type-specific 2D experiments required for the analysis of the main chain signals and side chain signals are specified corresponding to the primary sequence of the polypeptide chain.

9. The system of claim 3, wherein the NMR experiments for the analysis of the main chain signals and side chain signals comprise a combination of 2D and 3D experiments.

10. The system of claim 1, further comprising an evaluation of the NMR spectra is carried out in order to determine chemical shifts and coupling constants.

11. The system of claim 10, wherein the evaluation is carried out starting from the assignment of the NMR signals to the various spin systems of the polypeptide chain.

12. The system of claim 10, wherein the chemical shifts are collated and checked for accuracy.

13. The system of claim 12, wherein the shifts are collated and checked starting from the assignment of the NMR signals to the various spin systems of the polypeptide chain.

14. The system of claim 1, wherein the set of NMR spectra comprise spectra of the NOESY type provides information on the distances of the various nuclei of the polypeptide chain.

15. The system of claim 14, wherein the NOESY type evaluation provides information on the distances of the various nuclei of the polypeptide chain.

16. The system of claim 14, wherein the assignment of the NOESY type NMR spectra to the various nuclei of the polypeptide chain is carried out on the basis of the chemical shifts determined for the nuclei.

17. The system of claim 10, wherein results obtained from the evaluation of the NMR spectra serve as input quantities for structure calculation programs.

18. The system of claim 1, wherein the cross-signal pattern search masks comprise predefined signal search regions, wherein the NMR signals within the region boundaries provides an increased probability that the signal pattern defined by the cross-signal pattern search mask is present.

19. The system of claim 1, wherein the cross-signal pattern search masks comprise a number of predefined empty regions, wherein the absence of NMR signals within the region boundaries provides an increased probability that the signal pattern defined by the cross-signal pattern search mask is present.

20. The system of claim 1, wherein the cross-signal pattern search masks comprise threshold values and search regions determined by iteration, starting from the expected number of NMR signals in the spectra.

21. The system of claim 1, wherein the cross-signal pattern search mask comprises a plurality of sub-search masks for the analysis of the various NMR spectra of the recorded set of NMR spectra.

22. A process for the automated analysis of a set of NMR spectra, recorded for a polypeptide chain, comprising:
a) selecting a cross-signal pattern search mask from a library of cross-signal pattern search masks, wherein the mask detects a NMR signal of a fragment of the polypeptide chain, and wherein the selection of the required cross-signal pattern search masks is made corresponding to the fragments contained in the primary sequence;
b) executing a pattern recognition by correlating the various selected cross-signal pattern search masks with the set of NMR spectra; and
c) assigning the NMR signal to the various spin systems of the polypeptide chain corresponding to the result of the pattern recognition carried out in step b).

23. The process of claim 22, wherein the fragment of the polypeptide chain comprises two or three contiguous amino acids.

24. The process of claim 22, wherein the set of NMR spectra comprises NMR experiments for the analysis of the main chain signals and NMR experiments for the analysis of the side chain signals.

25. The process of claim 14, wherein the NMR experiments used for the analysis of the main chain signals comprises 3D experiments.

26. The process of claim 25, wherein the 3D experiments of the types CBCA (CO) NNH, CBCANNH, HA (CO)NNH, HANNH, HAHB (CO)NNH, HAHBNNH, HN (CA) CO, HNCO, HN(CO) CA and HNCA type experiments.

27. The process of claim 24, wherein the NMR experiments used for the analysis of the side chain signals comprise experiments of the types HCCH-COSY, HCCH-TOCSY and HCC(CO)NHTOCSY.

28. The process of claim 24, wherein the NMR experiments used for the analysis of the main chain signals and side chain signals comprise amino acid type-specific 2D experiments that are selective for an amino acid type or for a group of amino acid types.

29. The process of claim 28, wherein the amino acid type-specific 2D experiments required for the analysis of the main chain signals and side chain signals are specified corresponding to the primary sequence of the polypeptide chain.

30. The process of claim 22, wherein a combination of 2D and 3D experiments is used for the analysis of the main chain signals and side chain signals.

31. The process of claim 22, further comprising evaluating the NMR spectra to determine chemical shifts and coupling constants.

32. The process of claim 31, wherein the evaluation is carried out starting from the assignment of the NMR signals to the various spin systems of the polypeptide chain.

33. The process of claim 31, wherein chemical shifts are collated.

34. The process of claim 33, wherein the shifts are collated starting from the assignment of the NMR signals to the various spin systems of the polypeptide chain and are checked for their correctness.

35. The process of claim 22, wherein the set of NMR spectra comprises spectra of the NOESY type.

36. The process of claim 35, wherein the assignment of the NMR spectra of the NOESY type to the various nuclei of the polypeptide chain is carried out starting from the chemical shifts determined for the various nuclei.

37. The process of claim 31, wherein the quantities obtained in the evaluation of the NMR spectra are used as input quantities for structure calculation programs.

38. Analysis system for the automated analysis of a set of NMR spectra that has been recorded for a polypeptide chain comprising n amino acids, with a library of cross-signal pattern search masks, in which a cross-signal pattern search mask is provided for the specific detection of the NMR signals of a fragment of the investigated polypeptide chain, with means for selecting the cross-signal pattern search masks required for the analysis from the library of cross-signal pattern search masks corresponding to the primary sequence of the polypeptide chain, the said means selecting the associated cross-signal pattern search mask for each fragment contained in the primary sequence, with means for the pattern recognition, which combine the various results of the cross-signal pattern search masks selected corresponding to the primary sequence of the polypeptide chain and correlate them to the set of NMR spectra, and with means for assigning the NMR signals to the various spin systems of the polypeptide chain corresponding to the result of the pattern recognition; wherein n is an integer greater than or equal to 2.
